(19) United States
(12) Patent Application Publication
(10) Pub. No.: US 2019/0120760 A1
(43) Pub. Date: Apr. 25, 2019

(54) A SYSTEM AND METHOD FOR FLUID ANALYSIS

(71) Applicant: AGENCY FOR SCIENCE, TECHNOLOGY AND RESEARCH, Singapore (SG)

(72) Inventors: Szu Cheng LAI, Singapore (SG); Lin KE, Singapore (SG); Nan ZHANG, Singapore (SG)

(73) Assignee: AGENCY FOR SCIENCE, TECHNOLOGY AND RESEARCH, Singapore (SG)

(21) Appl. No.: 16/090,166

(22) PCT Filed: Mar. 30, 2017
(86) PCT No.: PCT/SG2017/050174

§ 371 Date: Sep. 28, 2018

(30) Foreign Application Priority Data
Mar. 30, 2016 (SG) 10201602512S

(51) Int. Cl. G01N 21/51; G01N 33/04; G01N 33/14

(52) U.S. Cl. CPC G01N 21/51; G01N 33/04; G01N 2201/061; G01N 2201/0633; G01N 33/14

(57) ABSTRACT

In a first aspect, a system for analysing a fluid sample is provided, comprises: a container configured to receive the fluid sample; a first light source optically coupled to the container and configured to project a first light having a first wavelength; a second light source optically coupled to the container and configured to project a second light having a second wavelength different from the first wavelength; one or more photo-sensors optically coupled to the container and configured to analyse the fluid sample in response to a first scattered light generated in response to projecting the fluid sample with the first light source and a second scattered light generated in response to projecting the fluid sample with the second light source. In a preferred embodiment, the nutritious compositions (such as the concentration of fats and protein) of milk samples are identified based on the scattering of the first and second light source by the milk sample.

### PRIORITY CLAIM

This application claims priority to Singapore Patent Application No. 10201602512S filed on 30 Mar. 2016, the content of which is incorporated herein by reference in its entirety for all purpose.

### FIELD OF INVENTION

The present invention relates broadly, but not exclusively, to methods and systems for analysing a fluid sample, more particularly, methods and systems for analysing a milk sample.

### BACKGROUND

Milk adulteration is a pervasive problem plaguing the dairy industries of developing countries, in particular India and China. According to a 2012 survey conducted by Food Safety and Standards Authority of India, FSSAI, over 68% of the milk products in India did not conform to the national food regulation standards due to adulteration practices. In China, on the other hand, milk adulterations are still prevalent today even after the intense public outcry against the melamine-tainted milk scandal in 2008. It is, therefore, important to contribute to the enforcement of milk standards by providing a reliable system and method for milk analysis including the identification of the types of the milk, measurement of the nutritious compositions of the milk as well as rapid screening for possible adulterations on the milk.

The conventional chemical analytical techniques for measuring the nutritious compositions of the milk, such as those of Babcock, Gerber, Kjeldahl or Dumas, are encumbered with tedious procedures involving the addition of chemical reagents, heating, titration and butyrometric measurements. For a faster analysis process, the dairy industry is adopting the acoustic technique based on ultrasonic reflection of the suspension particles in the milk. The acoustic technique, however, is considered to be less accurate as the measurement is susceptible to errors induced by the floating fat films and temperature of the milk sample.

More recently, optical techniques based on infra-red (IR) or near-infra-red (NIR) absorbance for measuring the milk nutritious compositions have been developed. Such techniques enable rapid and precise measurements on the milk, but are also costly to implement due to the intricacies of the electro-optical setup in the measurement system. Moreover, these techniques are highly sensitive to the extent that the results can be inadvertently influenced by the slight molecular differences in the natural compositions of the milk. As a result, the measurements can appear to be inconsistent when the techniques are deployed on the milk produced from different breeds of the cattle.

Alternatively, milk analysis can also be performed with another optical technique based on the scattering effect of light of a specific wavelength by the milk suspension particle. This technique, however, requires the addition of chemical reagents for dissolving specific components in the milk sample, so as to isolate the light scattering effect attributed to the un-dissolved component for its measurement. For instance, in the reported prior arts, diamine tetraacetic acid (EDTA) is added to the milk sample to dissolve the protein particles so as to isolate the light scattering effect by the fat particles for the measurement of fat concentration. However, the use of chemical reagents is considered undesirable as these chemical reagents are often expensive and polluting to the environment.

Accordingly, what is needed is a system and method for analyzing a fluid sample, particularly a milk sample, that seeks to address some of the above problems. Furthermore, other desirable features and characteristics will become apparent from the subsequent detailed description and the appended claims, taken in conjunction with the accompanying drawings and this background of the disclosure.

### SUMMARY OF INVENTION

In a first aspect, a system for analysing a fluid sample is provided, comprising:
a container configured to receive the fluid sample;
a first light source optically coupled to the container and configured to project a first light having a first wavelength;
a second light source optically coupled to the container and configured to project a second light having a second wavelength different from the first wavelength;

one or more photo-sensors optically coupled to the container and configured to analyse the fluid sample in response to a first scattered light generated in response to projecting the fluid sample with the first light source and a second scattered light generated in response to projecting the fluid sample with the second light source.

In one embodiment, the one or more photo-sensors are configured to be positioned perpendicular to projection paths of the first light and the second light.

In one embodiment, the first light source and the second light source are collimated light sources.

In one embodiment, the first light source and the second light source are laser sources.

In one embodiment, the first wavelength of the first light is red, and the second wavelength of the second light is blue.

In one embodiment, the fluid sample includes at least one of milk, diluents and other reagents.

In one embodiment, the diluents and other reagents include water, organic solvents and inorganic solvents.

In one embodiment, the one or more photo-sensors are configured to analyze the fluid sample based on a differential scattering in response to the first scattered light and the second scattered light.

In one embodiment, the one or more photo-sensors are configured to identify a type of the fluid sample.

In one embodiment, the one or more photo-sensors are configured to identify components of the fluid sample.

In one embodiment, the one or more photo-sensors are configured to identify a concentration of fats and protein of the fluid sample.

In another aspect, a method for analysing a fluid sample is provided, comprising: projecting a first light onto the fluid sample, the first light having a first wavelength;

projecting a second light onto the fluid sample, the second light having a second wavelength different from the first wavelength; and

analysing the fluid sample in response to a first scattered light generated in response to projecting the fluid sample with the first light source and a second scattered light generated in response to projecting the fluid sample with the second light source.

In one embodiment, the step of analysing the fluid sample is performed perpendicularly to projection paths of the first light and the second light.

In one embodiment, the first wavelength of the first light is red, and the second wavelength of the second light is blue.

In one embodiment, the fluid sample includes milk, diluents and other reagents.

In one embodiment, the step of analysing is performed based on a differential scattering in response to the first scattered light and the second scattered light.

In one embodiment, the step of analysing is performed to identify a type of the fluid sample.

In one embodiment, the step of analysing is performed to identify components of the fluid sample.

In one embodiment, the diluents and other reagents include water, organic solvents and inorganic solvents.

In one embodiment, the step of analysing is performed to identify a concentration of fats and protein of the fluid sample

### BRIEF DESCRIPTION OF DRAWINGS

Embodiments of the invention will be better understood and readily apparent to one of ordinary skill in the art from the following written description, by way of example only, and in conjunction with the drawings, in which:

FIG. 1 shows a system according to one embodiment.

FIG. 2 shows a system according to another embodiment.

FIG. 3 shows a method for analysing the fluid sample according to one embodiment.

FIG. 4 shows a method for analysing the fluid sample according to another embodiment.

FIG. 5 shows a system for analysing the fluid sample according to one embodiment.

FIG. 6 shows a table tabulating nutritious compositions of milk samples and outputs of the photo-sensor for measuring the scattering intensities of the red and blue laser.

FIG. 7 shows a plot of milk type identity metric versus protein-fat proportion.

FIG. 8 shows a table showing calibration equations for different milk types.

FIG. 9 shows a computation results for fat and protein concentrations.

FIG. 10 shows a schematic diagram of a computer system suitable for use in executing the method depicted in FIG. 3

### DETAILED DESCRIPTION

Embodiments of the present invention will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “capturing”, “estimating”, “overfilling”, “analysing”, “projecting”, “retrofitting”, “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM mobile telephone system. The computer program when loaded and executed on such a computer effectively results in an apparatus that implements the steps of the preferred method.

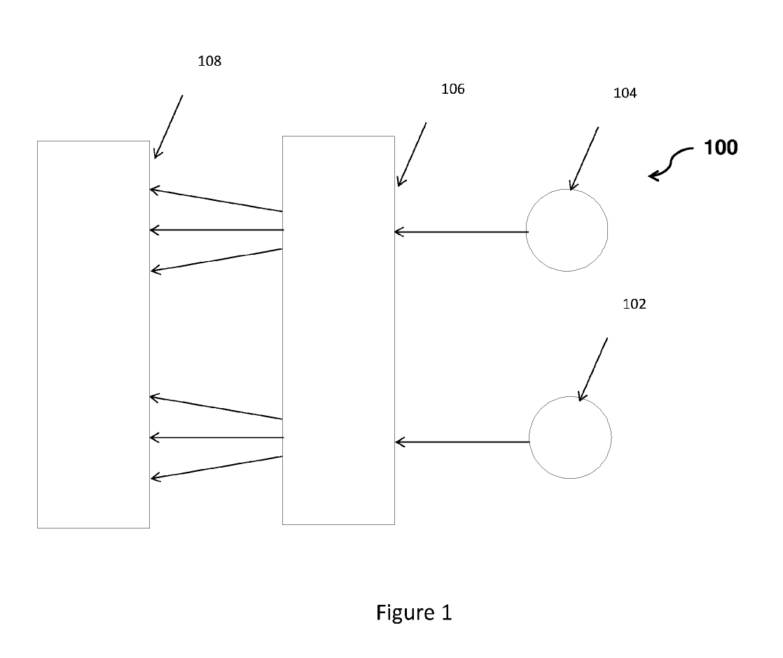

FIG. 1 shows a system according to one embodiment. As shown, there is a system 100 for analysing a fluid sample. The system 100 comprises a container 106 configured to receive the fluid sample, a first light source 102 optically coupled to the container 106 and configured to project a first light having a first wavelength, a second light source 104 optically coupled to the container 106 and configured to project a second light having a second wavelength different from the first wavelength. Further, the system 100 includes one or more photo-sensors 108 optically coupled to the container 106 and configured to analyse the fluid sample in response to a first scattered light generated in response to projecting the fluid sample with the first light source 102 and a second scattered light generated in response to projecting the fluid sample with the second light source 104.

Embodiments of the invention relate to an optical system and method for fluid analysis, particularly milk analysis based on the scattering of light of at least two different wavelengths into a fluid sample. That is, the lights that are projected into the fluid sample are of two different wavelengths. It is to be understood that the lights of different wavelengths are scattered differently by particles in the fluid sample. In addition, certain type of particles can scatter a specific wavelength of light more than another type of the particles. The difference in the scattering effects of a specific wavelength by the different types of the particles, or hereby known as the differential scattering effect, is caused by a different degree of deflection by the particles of different sizes.

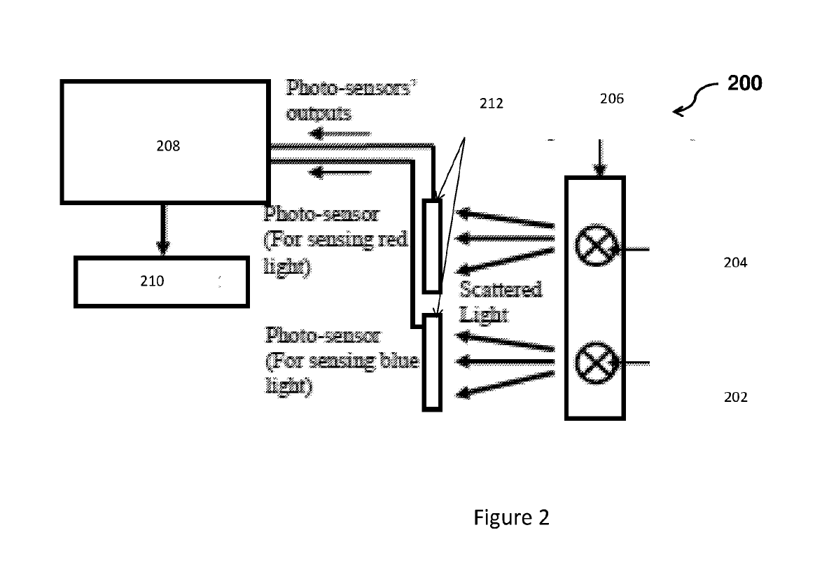

FIG. 2 shows a system according to one embodiment. The system 200 includes an optically transparent container 206 which include, among other things, a cuvette and a glass bottle, and configured to hold the fluid sample. The container 206 may have one side facing a first light source 202 and a second light source 204 and another opposing side facing one or more photo-sensors 212.

The one or more photo-sensors 212 may be oriented at an angle to the path of the lights projected by the first light source 202 and the second light source 204. In an embodiment, the one or more photo-sensors 212 may be oriented perpendicular to the path of the lights projected by the first light source and the second light source.

In various embodiments, there may be one photo-sensor 212 while in other embodiments, there may be more than one photo-sensor 212. That is, there may be two or more photo-sensors 212; one photo-sensor 212 may be configured for measuring the first scattered light generated in response to the projecting the fluid sample with the first light source 202 and a different photo-sensor 212 may be configured for measuring the second scattered light generated in response to the projecting the fluid sample with the second light source 204.

The first and second light sources 202, 204 may be configured to project collimated light beams. Additionally or alternatively, the first and second light sources 202, 204 may be configured to minimise the dispersion of light during propagation or projection to the fluid sample received in the container 206.

In various embodiments below, the first wavelength of the first light is preferably red (approximately in the range of 620-750 nm) or any other similar light and the second wavelength of the second light is preferably blue (approximately in the range of 360-490 nm) or any other similar light.

The fluid sample may include milk or milk added with diluents such as water, solvents (organic or inorganic), and reagents. The diluents may include those for diluting the milk's concentration in the sample, or for dissolving, breaking down or precipitating any components in the fluid sample.

In embodiments where the fluid sample includes milk, the milk sample may include milks of different types or variations. The types may be defined by the animals or origins (such as cows, goats or buffalos), or by the nutritious compositions (such as the proportions of fats, protein and lactose). It is further preferred that the milk in the milk sample is homogenised.

In the system 200, the one or more photo-sensors 212 may be operationally coupled to a micro-processor 208 which includes an algorithm for analysing the fluid sample or milk sample. In one embodiment, the algorithm seeks to identify the milk types based on the scattering of the first and second light source 202, 204 by the milk sample as measured by the photo-sensor 212. Each of the photo-sensors is also aligned to the same height level as each of the light beams, so as to effectively capture the scattered light from each of the light source. The photo-sensors are also configured to provide an electrical signal in linear with the intensity of the scattered lights.

More specifically, the identification of the milk types may be based on the differential scattering effects between the first and second light sources 202,204 projected into the milk sample. Such differential scattering effects between the first and second light sources 202, 204 may be described by mathematical metrics, such as the ratio of the scattering of the first light to that of the second light. The milk analysis algorithm embedded in the micro-processor 208 includes a protocol for identifying the milk types as defined by the animals of origin (such as cows, goats or buffalos), or by the nutritious compositions (such as the proportions of fat, protein and lactose). The identification of the milk types is based on the differential scattering effects between the red and blue lasers projected into the milk sample, as defined by the ratio of the scattering of the blue light to that of the red light. Correspondingly, these mathematical metrics can assume the milk identity metrics. Correspondingly, the identification is based on a milk identity metric, M, which relates the ratio of the scattering of the blue light to that of the red light to a specific milk type, whereby M is in the form of:

$$M = \frac{Bs}{Rs} \qquad ( 1 )$$

where Bs and Rs are the scattering intensity of blue and red light respectively.

In another embodiment, the algorithm seeks to measure the nutritious compositions of the milk based on the scattering of the first and second light source 202, 204 by the milk sample as measured by the photo-sensor 212. For example, the fat and protein concentrations can be derived based on the set of calibrated equations which takes the forms of:

R_{E}=f₁(X_{F},X_{P}) (2a)

Bₛ=f₂(X_{F},X_{P}) (2b)

where X_{F} and X_{P} are the concentrations of fat and protein respectively

The calibrated equations can be modeled by means of data-fitting the intensities of the scattered lights against the actual nutritious composition from milk samples of different concentrations.

The milk analysis algorithm further includes a protocol for detecting adulteration or spoilage by evaluating if the milk identity metrics, M, or the measurements on the nutritious compositions fall outside the acceptable range.

More specifically, the measurements on the nutritious compositions are based on a set of calibrated equations relating the scatterings of the first and second light source 202, 204 with the nutritious compositions. The calibrated equation may be modeled by means of data-fitting the intensities of the scattered lights against the actual nutritious composition from milk samples of different concentrations.

In another embodiment, the algorithm seeks to detect cases associating with adulteration or spoilage based on the scattering of the first and second light source 202, 204 by the milk sample as measured by the photo-sensor 212. More precisely, the detection of adulteration or spoilage is performed by evaluating if the milk identity metrics as devised by the various embodiments or the measurements on the compositions fall outside the acceptable range.

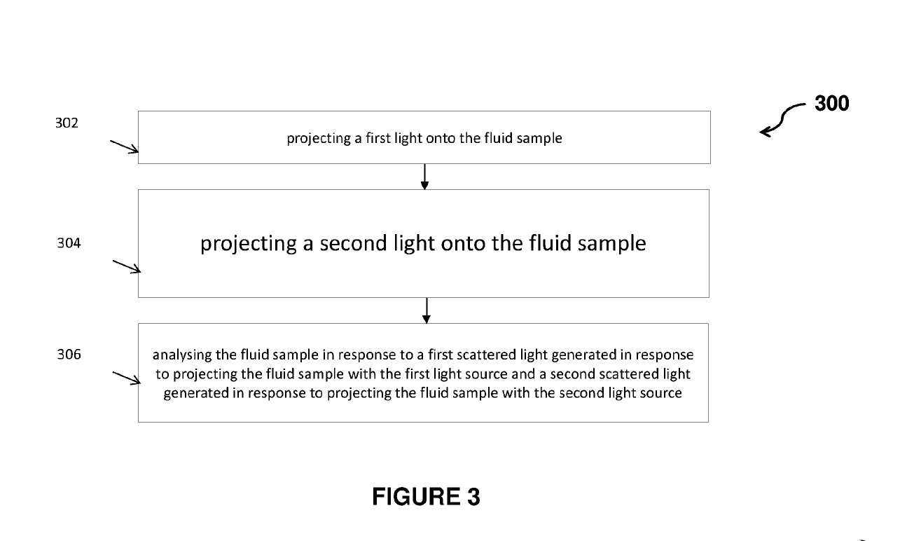

FIG. 3 shows a method for analysing a fluid sample according to an embodiment. The method 300 broadly includes:
step 302: projecting a first light onto the fluid sample, the first light having a first wavelength
step 304: projecting a second light onto the fluid sample, the second light having a second wavelength different from the first wavelength
step 306: analysing the fluid sample in response to a first scattered light generated in response to projecting the fluid sample with the first light source and a second scattered light generated in response to projecting the fluid sample with the second light source.

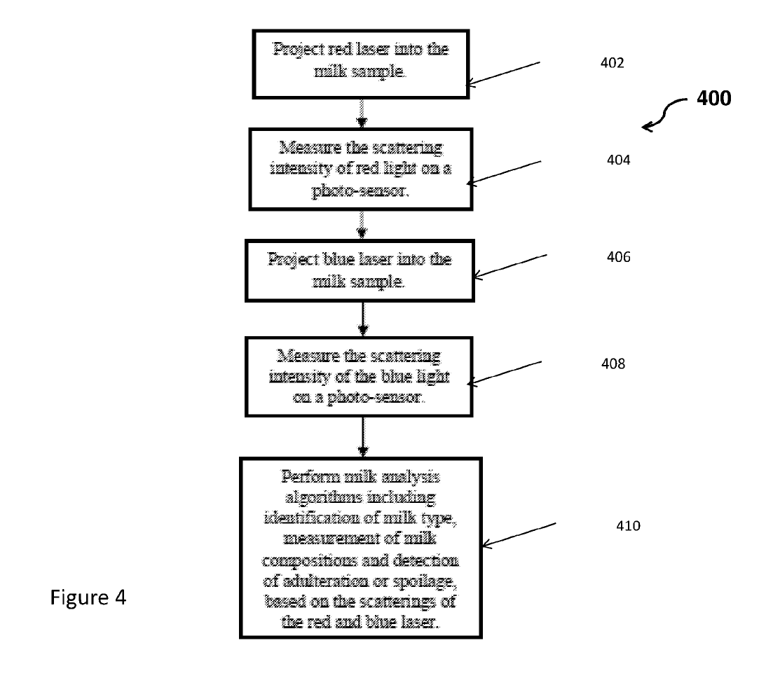

FIG. 4 shows a method for analysing the fluid sample according to one embodiment. At operation 402, a red light is projected into the milk sample. At operation 404, the scattering intensity of the red light that is projected into the milk sample is measured. At operation 406, a blue light is projected into the milk sample. At operation 408, the scattering intensity of the blue light that is projected into the milk sample is measured. At operation 410, milk analysis is performed using algorithm including identification of milk type, measurement of milk compositions and detection of adulteration or spoilage based on the scatterings of blue and red lights.

Experimental Set-Up

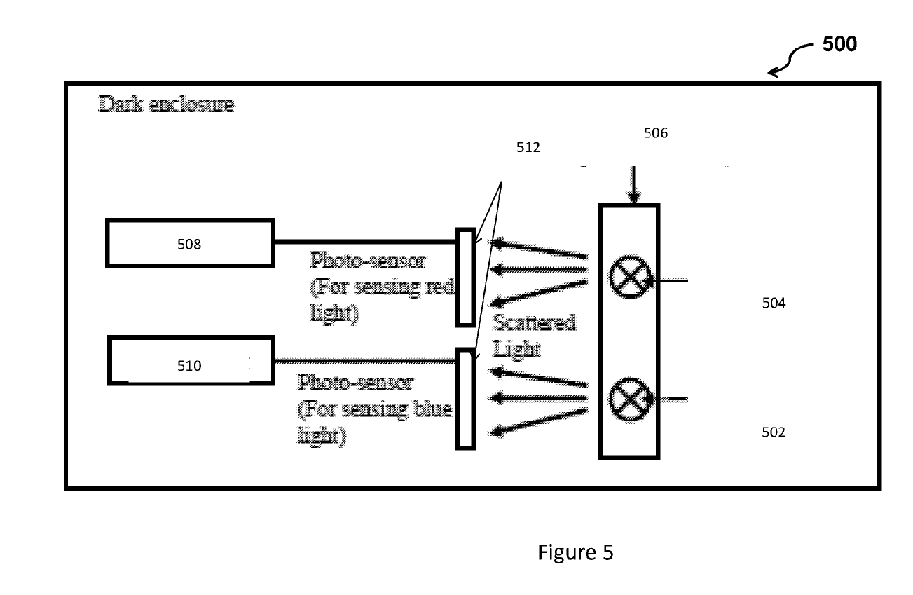

To verify the technical feasibility of the various embodiments, milk samples of various fat-protein proportions and concentrations were prepared and subjected to red and blue light scattering measurements in the system setup similar to the present invention, as illustrated in FIG. 5.

FIG. 5 shows a system 500 according to one experiment set up. As shown, there is a system 500 for analysing a fluid sample. The system 500 comprises a container 506 configured to receive the fluid sample, a first light source 502 optically coupled to the container 506 and configured to project a first light having a first wavelength, a second light source 504 optically coupled to the container 506 and configured to project a second light having a second wavelength different from the first wavelength. Further, the system 500 includes one or more photo-sensors 512 optically coupled to the container 506 and configured to analyse the fluid sample in response to a first scattered light generated in response to projecting the fluid sample with the first light source 502 and a second scattered light generated in response to projecting the fluid sample with the second light source 504.

Sample Preparation and Light Measurements

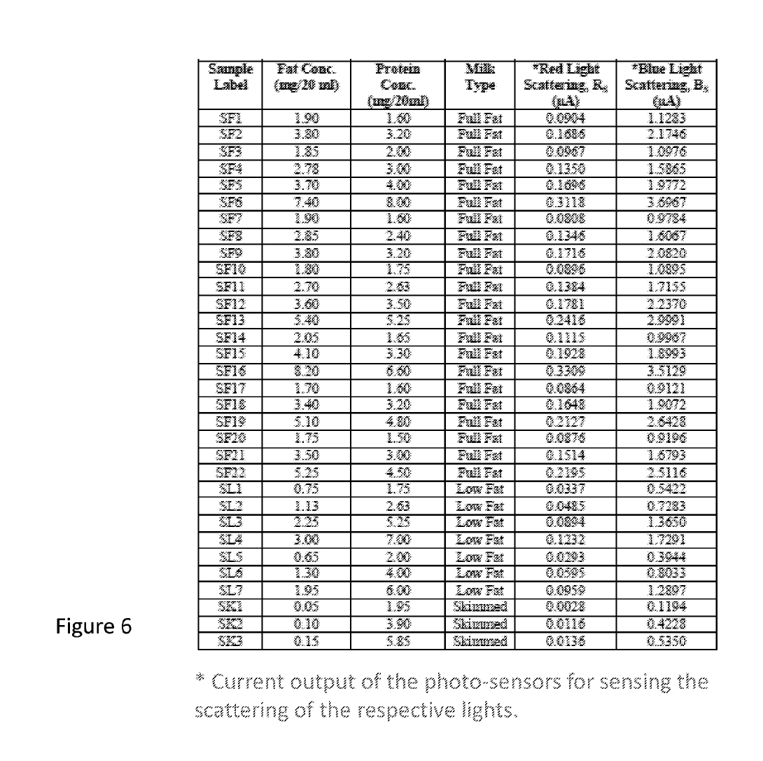

Fresh milk of various compositions including Full-fat, Low-fat and Skimmed milk were used as the experimental samples. The milk samples were further diluted by dispensing the fresh milks of various volumes ranging from 50-200 ul in de-ionized water of volume of 20 millimeters less the volume of the milk dispensed. The nutritious composition of the resulting samples and the respective measurements on the red and blue light scatterings were tabulated in the table shown in FIG. 6.

Identification of Milk Types and Milk Identity Metrics

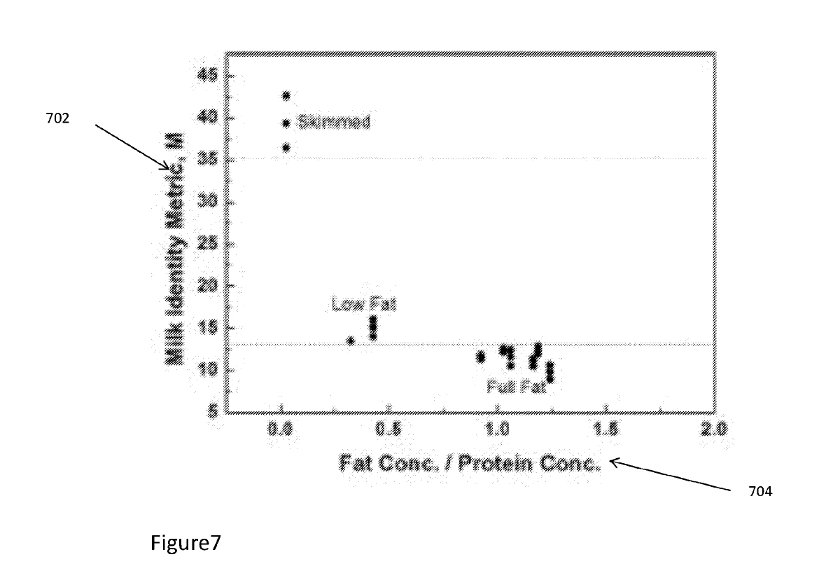

The ratios of the scatterings of the blue light to that of the red laser, Bs/Rs, describing the differential scattering effects between the 2 wavelengths were computed. It was observed that under the same milk types (as defined by Full-fat, Low-fat and Skimmed) the above ratios clustered within a defined range even as the concentrations of the sample varied widely, as illustrated in FIG. 7 The ratios of the blue to red light scattering (as denoted by M in FIG. 7), therefore, effectively serves as the milk identity metric for identifying milk of different fat-protein proportion.

Measurements on Milk Compositions

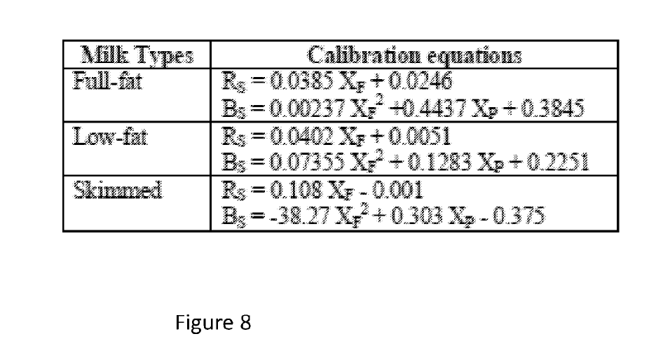

By means of data-fitting on the table shown in FIG. 6, the calibrated equations relating fat and protein concentrations with the scatterings of red and blue lasers can be modeled. The calibration equations for each of the milk types are summarized in a table shown in FIG. 8, where X_{F} and X_{P} are the concentrations of fat and protein, respectively.

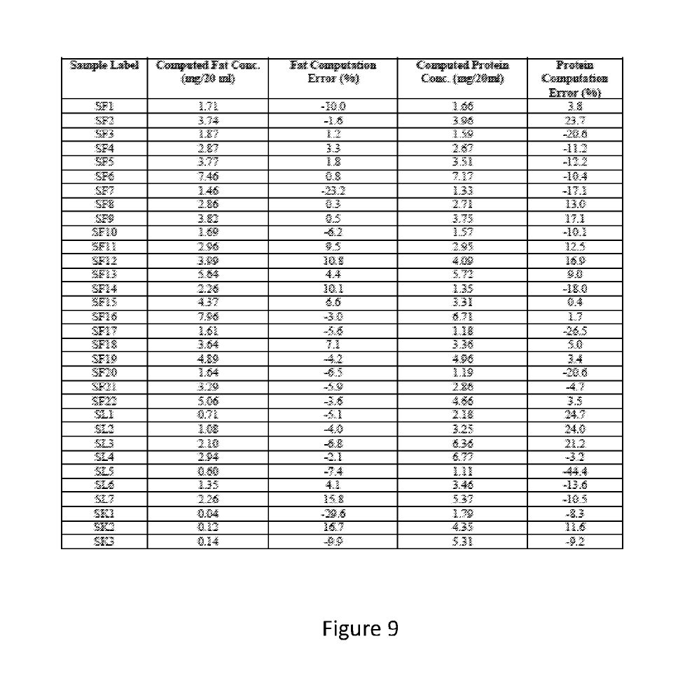

The red and blue light scatterings data shown in the table of FIG. 6 are re-fitted into the calibration equations in FIG. 8 for the respective milk types. The results, as compiled in table of FIG. 9, shows that the fat and protein measurements are generally close to the actual concentrations, with an error typically under +/−10% for fat and +/−20% for protein.

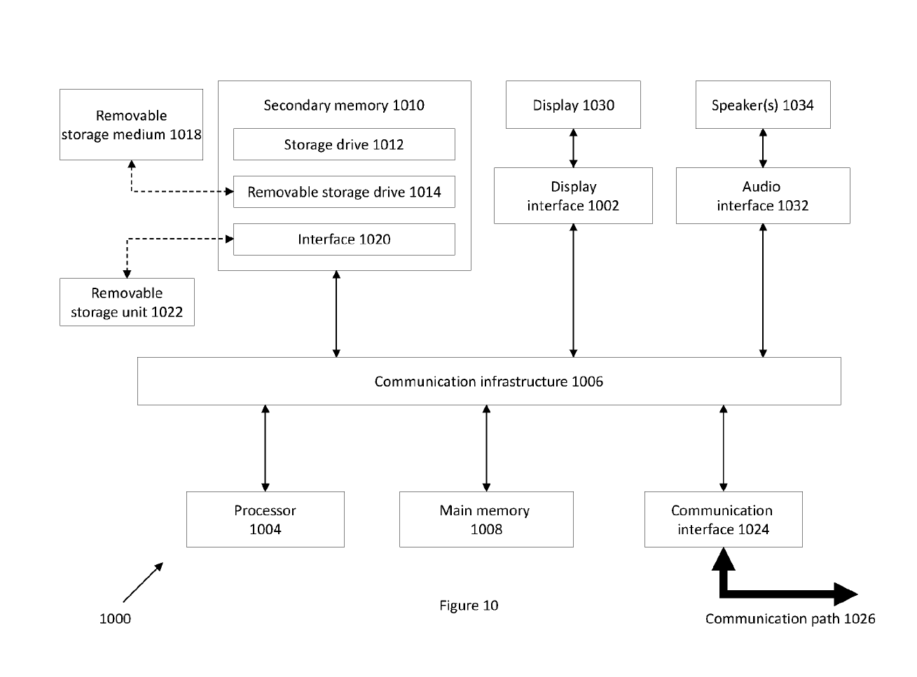

FIG. 10 depicts an exemplary computer/computing device 1000, hereinafter interchangeably referred to as a computer system 1000, where one or more such computing devices 1000 may be used to facilitate execution of the above-described method for providing a travel recommendation to a user. In addition, one or more components of the computer system 1000 may be used to realize the computer 1000. The following description of the computing device 1000 is provided by way of example only and is not intended to be limiting.

As shown in FIG. 10, the example computing device 1000 includes a processor 1004 for executing software routines. Although a single processor is shown for the sake of clarity, the computing device 1000 may also include a multi-processor system. The processor 1004 is connected to a communication infrastructure 1006 for communication with other components of the computing device 1000. The communication infrastructure 1006 may include, for example, a communications bus, cross-bar, or network.

The computing device 1000 further includes a main memory 1008, such as a random access memory (RAM), and a secondary memory 1010. The secondary memory 1010 may include, for example, a storage drive 1012, which may be a hard disk drive, a solid state drive or a hybrid drive and/or a removable storage drive 1014, which may include a magnetic tape drive, an optical disk drive, a solid state storage drive (such as a USB flash drive, a flash memory device, a solid state drive or a memory card), or the like. The removable storage drive 1014 reads from and/or writes to a removable storage medium 1018 in a well-known manner. The removable storage medium 1018 may include magnetic tape, optical disk, non-volatile memory storage medium, or the like, which is read by and written to by removable storage drive 1014. As will be appreciated by persons skilled in the relevant art(s), the removable storage medium 1018 includes a computer readable storage medium having stored therein computer executable program code instructions and/or data.

In an alternative implementation, the secondary memory 1010 may additionally or alternatively include other similar means for allowing computer programs or other instructions to be loaded into the computing device 1000. Such means can include, for example, a removable storage unit 1022 and an interface 1020. Examples of a removable storage unit 1022 and interface 1020 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 1022 and interfaces 1020 which allow software and data to be transferred from the removable storage unit 1022 to the computer system 1000.

The computing device 1000 also includes at least one communication interface 1024. The communication interface 1024 allows software and data to be transferred between computing device 1000 and external devices via a communication path 1026. In various embodiments of the inventions, the communication interface 1024 permits data to be transferred between the computing device 1000 and a data communication network, such as a public data or private data communication network. The communication interface 1024 may be used to exchange data between different computing devices 1000 which such computing devices 1000 form part an interconnected computer network. Examples of a communication interface 1024 can include a modem, a network interface (such as an Ethernet card), a communication port (such as a serial, parallel, printer, GPIB, IEEE 1394, RJ25, USB), an antenna with associated circuitry and the like. The communication interface 1024 may be wired or may be wireless. Software and data transferred via the communication interface 1024 are in the form of signals which can be electronic, electromagnetic, optical or other signals capable of being received by communication interface 1024. These signals are provided to the communication interface 1024 via the communication path 1026.

As shown in FIG. 10, the computing device 1000 further includes a display interface 1002 which performs operations for rendering images to an associated display 1030 and an audio interface 1032 for performing operations for playing audio content via associated speaker(s) 1034.

As used herein, the term “computer program product” may refer, in part, to removable storage medium 1018, removable storage unit 1022, a hard disk installed in storage drive 1012, or a carrier wave carrying software over communication path 1026 (wireless link or cable) to communication interface 1024. Computer readable storage media refers to any non-transitory, non-volatile tangible storage medium that provides recorded instructions and/or data to the computing device 1000 for execution and/or processing. Examples of such storage media include magnetic tape, CD-ROM, DVD, Blu-ray™ Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a SD card and the like, whether or not such devices are internal or external of the computing device 1000. Examples of transitory or non-tangible computer readable transmission media that may also participate in the provision of software, application programs, instructions and/or data to the computing device 1000 include radio or infra-red transmission channels as well as a network connection to another computer or networked device, and the Internet or Intranets including e-mail transmissions and information recorded on Websites and the like.

The computer programs (also called computer program code) are stored in main memory 1008 and/or secondary memory 1010. Computer programs can also be received via the communication interface 1024. Such computer programs, when executed, enable the computing device 1000 to perform one or more features of embodiments discussed herein. In various embodiments, the computer programs, when executed, enable the processor 1004 to perform features of the above-described embodiments. Accordingly, such computer programs represent controllers of the computer system 1000.

Software may be stored in a computer program product and loaded into the computing device 1000 using the removable storage drive 1014, the storage drive 1012, or the interface 1020 Alternatively, the computer program product may be downloaded to the computer system 1000 over the communications path 1026. The software, when executed by the processor 1004, causes the computing device 1000 to perform functions of embodiments described herein.

It is to be understood that the embodiment of FIG. 10 is presented merely by way of example. Therefore, in some embodiments one or more features of the computing device 1000 may be omitted. Also, in some embodiments, one or more features of the computing device 1000 may be combined together. Additionally, in some embodiments, one or more features of the computing device 1000 may be split into one or more component parts.

Advantageously, a systematic and accurate approach is proposed that is based on the scattering of lights of least two different wavelengths projected into the fluid sample. Based on the light scattering measurements under at least two wavelengths, various embodiments seek to provide a cost-effective, rapid and clean system and method for quantitative and qualitative milk analysis including the identification of the types of the milk, measurement of the nutritious compositions of the milk as well as rapid screening for possible adulterations on the milk. The proposed approach does not require the addition of chemical reagents in order to work. The approach that is described in the various embodiments above enable rapid and precise measurements on the milk sample and yet cost-effective to implement due to the simple setup in the measurement system.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1-20. (canceled)

21. A system for analysing a milk sample, comprising:
a container configured to receive the milk sample;
a first light source optically coupled to the container and configured to project a first light having a first wavelength;
a second light source optically coupled to the container and configured to project a second light having a second wavelength different from the first wavelength;
one or more photo-sensors optically coupled to the container; and
a processing means coupled to the one or more photo-sensors, the processing means configured to analyse the milk sample in response to a first scattered light generated in response to projecting the milk sample with the first light source and a second scattered light generated in response to projecting the milk sample with the second light source to determine at least fat and protein compositions of the milk sample.

22. A system according to claim 21, wherein the one or more photo-sensors are configured to be positioned perpendicular to projection paths of the first light and the second light.

23. A system according to claim 21, wherein the first light source and the second light source are collimated light sources.

24. A system according to claim 23, wherein the first light source and the second light source are laser sources.

25. A system according to claim 21, wherein the first wavelength of the first light is red, and the second wavelength of the second light is blue.

26. A system according to claim 1, wherein the milk sample includes at least one of diluents and other reagents.

27. A system according to claim 26, wherein the diluents and other reagents include water, organic solvents and inorganic solvents.

28. A system according to claim 21, wherein the fat and protein compositions of the milk sample comprise absolute concentrations of the fat and protein compositions of the milk sample or relative proportions of the fat and protein compositions of the milk sample.

29. A system according to claim 21, wherein the processing means is further configured to analyse the milk sample in response to the first scattered light generated in response to projecting the milk sample with the first light source and the second scattered light generated in response to projecting the milk sample with the second light source to determine a milk identity metric, the milk identify metric identifying a milk type as defined by an animal of origin.

30. A method for analysing a milk sample, comprising:
projecting a first light onto the milk sample, the first light having a first wavelength;
projecting a second light onto the milk sample, the second light having a second wavelength different from the first wavelength;
detecting a first scattered light generated in response to projecting the milk sample with the first light source;
detecting a second scattered light generated in response to projecting the milk sample with the second light source; and
analysing the milk sample in response to the first scattered light and the second scattered light to determine at least fat and protein compositions of the milk sample.

31. The method according to claim 30, wherein the step of detecting the first scattered light and detecting the second scattered light are performed perpendicularly to projection paths of the first light and the second light, respectively.

32. The method according to claim 30, wherein the first wavelength of the first light is red, and the second wavelength of the second light is blue.

33. The method according to claim 30, wherein the milk sample includes at least one of diluents and other reagents.

34. The method according to claim 30, wherein the diluents and other reagents include water, organic solvents and inorganic solvents.

35. The method according to claim 30, wherein analysing the milk sample to determine at least the fat and protein compositions of the milk sample comprises analysing the milk sample to determine absolute concentrations of the fat and protein compositions of the milk sample or relative proportions of the fat and protein compositions of the milk sample.

36. The method according to claim 30, wherein analysing the milk sample comprises analysing the milk sample in response to the first scattered light and the second scattered light to determine a milk identity metric, the milk identify metric identifying a milk type as defined by an animal of origin.
